(19) United States
(12) Patent Application Publication
(10) Pub. No.: US 2024/0390216 A1
(43) Pub. Date: Nov. 28, 2024

(54) MASSAGE APPARATUS

(71) Applicant: NATIONAL UNIVERSITY OF CORPORATION CHIBA UNIVERSITY, Chiba-shi, Chiba (JP)

(72) Inventor: Masahiro Takei, Chiba-shi (JP)

(21) Appl. No.: 18/690,884

(22) PCT Filed: Oct. 26, 2022
(86) PCT No.: PCT/JP2022/039901

§ 371 Date: Mar. 11, 2024

(30) Foreign Application Priority Data
Oct. 26, 2021 (JP) 2021-174582

(51) Int. Cl. A61H 7/00

(52) U.S. Cl. CPC A61H 7/004; A61H 2201/5058

(57) ABSTRACT

A massage apparatus includes a plurality of pressers that press a subject, and an in-vivo measurer that measures a change in biological information of the subject due to pressing, wherein the in-vivo measurer has one or more sensors for electrical impedance tomography, and each of the sensors for electrical impedance tomography has four or more electrodes.

### TECHNICAL FIELD

The present invention relates to a massage apparatus.

The present application claims priority based on Japanese Patent Application No. 2021-174582 filed in Japan on Oct. 26, 2021, the contents of which are incorporated herein by reference.

### BACKGROUND ART

When the human body adopts a posture of a standing position for a long time, interstitial fluid of muscles or fat in the calf accumulates due to gravity, and edema is caused in the calves. Consequently, the human body has a sense of fatigue. In addition, in lymphedema which is a cancer sequela, interstitial fluid accumulates due to accumulation of lymphatic fluid and results in occurrence of edema. Therefore, there is a demand for a technology that causes interstitial fluid and lymphatic fluid to recirculate.

Massage is performed by externally applying pressure to a subject by using a massage ball or an air bag. Consequently, it is possible to not only knead muscles or fat of the subject, but also cause interstitial fluid, lymphatic fluid, and venous blood in the muscles or fat to recirculate.

For example, in limb massage, intermittent pneumatic compression (IPC) can be used to enable interstitial fluid, lymphatic fluid, and venous blood to effectively recirculate.

Intermittent pneumatic compression involves covering the body with a sleeve (bag) having a plurality of sections (chambers) and temporally and spatially adjusting the pneumatic pressure in the individual sections to conform to the curved surfaces of the limbs. For example, Patent Document 1 discloses an air massager including: a plurality of air bags that are wound around and perform an operation on a procedure target; a plurality of switching valve devices that individually supply and discharge air to and from these air bags via air supply pipes; a pneumatic compression source device connected to the switching valve device; a pressure sensor attached to the air supply pipes; a detection means that detects abnormality by sensing a pressure change state by the pressure sensor during operation; an alarm means that issues an alarm when the detection means detects occurrence of abnormality; and a time measurement/display means that measures and displays an elapsed time from a start time of the occurrence of the abnormality.

### CITATION LIST

### Patent Document

[Patent Document 1]
   Japanese Unexamined Patent Application, First Publication No. 2007-289321

### SUMMARY OF INVENTION

### Technical Problem

Recirculation of interstitial fluid, lymphatic fluid, and venous blood by massage has large individual differences and has large daily variations particularly for patients with lymphedema. In the air massager of Patent Document 1, a subject himself or herself cannot ascertain whether interstitial fluid, lymphatic fluid, and venous blood have been sufficiently recirculated by the massage. Therefore, the massage may be ended during the course thereof.

The present invention has been made in view of the above-described circumstances, and an object of the present invention is to provide a massage apparatus capable of measuring a change in distribution of interstitial fluid, lymphatic fluid, and venous blood in a living body due to massage.

### Solution to Problem

In order to solve the above problems, the present invention proposes the following means.
<1> A massage apparatus according to one aspect of the present invention includes:
a plurality of pressers that press a subject, and an in-vivo measurer that measures a change in biological information of the subject due to pressing, wherein
the in-vivo measurer has one or more sensors for electrical impedance tomography, and each of the sensors for electrical impedance tomography has four or more electrodes.
<2> The massage apparatus according to <1> may include two or more of the sensors for electrical impedance tomography.
<3> The massage apparatus according to <1> or <2> may include the pressers between the sensors for electrical impedance tomography.
<4> In the massage apparatus according to any one of <1> to <3> , each of the pressers may have one air bag.
<5> In the massage apparatus according to any one of <1> to <3> , each of the pressers may include two or more air bags, and the air bags are capable of applying respective different magnitudes of pressure.
<6> In the massage apparatus according to any one of <1> to <5> , the electrodes may be arranged at uniform intervals.
<7> In the massage apparatus according to any one of <1> to <6> , the in-vivo measurer may apply a current or a potential difference between the electrodes, measure a potential difference and a phase based on a current application/voltage measurement pattern in a case where the current is applied, and measure a current and a phase based on a voltage application/current measurement pattern in a case where the potential difference is applied between the electrodes.
<8> The massage apparatus according to <7> may further include: a Jacobian matrix calculator that calculates a Jacobian matrix of the subject based on the predetermined current application/voltage measurement pattern or voltage application/current measurement pattern, mesh coordinates obtained by dividing a contour of the subject, and coordinates of the respective electrodes; and an electrical property distribution calculator that calculates an electrical property distribution that is the biological information from the Jacobian matrix of the subject calculated by the Jacobian matrix calculator and the potential difference and the phase or the current and the phase measured by the in-vivo measurer.

<9> In the massage apparatus according to <8> , the Jacobian matrix calculator may calculate the Jacobian matrix by using machine learning.
<10> The massage apparatus according to > or <9> may further include a pressing controller that controls the pressure of each of the pressers based on the electrical property distribution.

### Advantageous Effects of Invention

According to the above-described aspect of the present invention, it is possible to provide a massage apparatus capable of measuring a change in distribution of interstitial fluid, lymphatic fluid, and venous blood in a living body by massage.

### BRIEF DESCRIPTION OF DRAWINGS

FIG. 1 is a schematic diagram of a massage apparatus according to a first embodiment.

FIG. 2 is a schematic diagram of a pressing measurer according to the first embodiment.

FIG. 3 is a schematic view of a sensor for electrical impedance tomography according to the first embodiment.

FIG. 4 is a diagram showing a current application/voltage measurement pattern. FIG. 5 is a diagram showing a data set I of known leg contours ∂Ω and a data set J of the Jacobian matrix of a known internal leg tissue Ω.

FIG. 6 is a flowchart of calculation of a Jacobian matrix J* in a Jacobian matrix calculator.

FIG. 7 is a schematic diagram of a massage apparatus according to a second embodiment.

FIG. 8 is a schematic diagram of a massage apparatus according to a third embodiment.

FIG. 9 is a schematic diagram of a pressing measurer according to the third embodiment.

FIG. 10 is a schematic diagram of a massage apparatus according to a fourth embodiment.

FIG. 11 is a schematic diagram of a pressing measurer according to the fourth embodiment.

FIG. 12 is a schematic view of a sensor for electrical impedance tomography according to the fourth embodiment.

FIG. 13 is a schematic view of a massage apparatus according to a fifth embodiment.

FIG. 14 is a schematic view of a sensor for electrical impedance tomography according to the fifth embodiment.

FIG. 15 is a schematic diagram of a massage apparatus used in an example.

FIG. 16 is a graph illustrating a change in pressure of an air bag during massage.

FIG. 17 is a diagram illustrating a temporal change of a conductivity distribution of a calf part of a subject obtained by electrical impedance tomography measurement during massage.

FIG. 18 is a diagram illustrating a temporal change of a conductivity distribution of a thigh of the subject obtained by the electrical impedance tomography measurement during massage.

FIG. 19 is a graph illustrating spatial mean normalized conductivity <σ> in the calf part and a relationship between pressure and time of each air bag.

FIG. 20 is a graph illustrating spatial mean normalized conductivity <σ> in the thigh and a relationship between pressure and time of each chamber.

### DESCRIPTION OF EMBODIMENTS

### First Embodiment

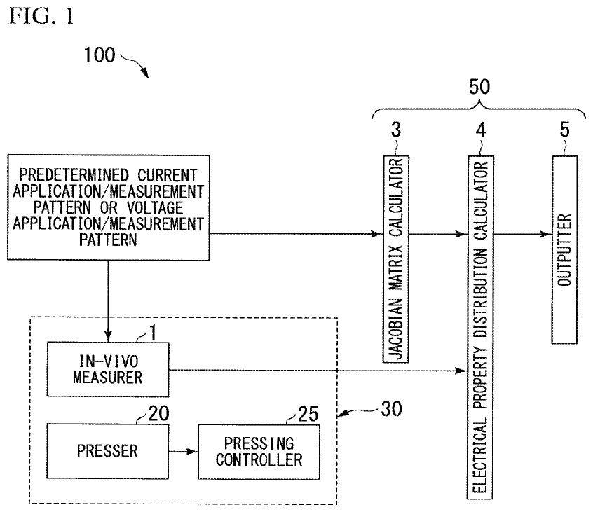

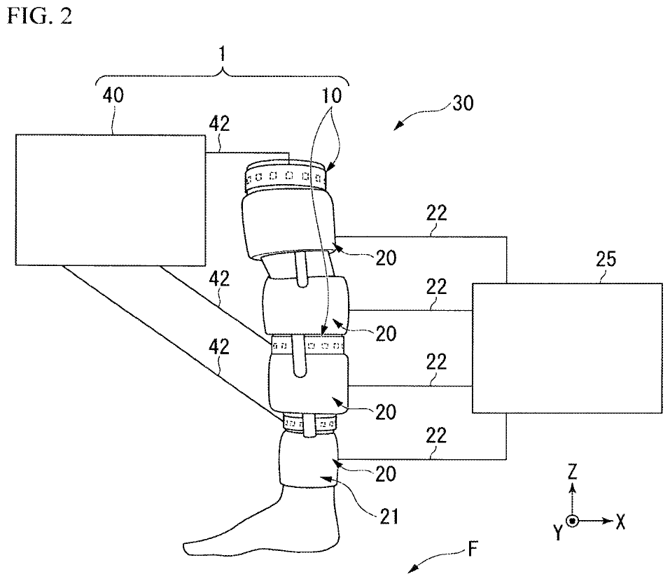

Hereinafter, a massage apparatus according to an embodiment of the present invention will be described with reference to the drawings. As illustrated in FIG. 1, a massage apparatus 100 includes a pressing measurer 30 and a measurement calculator 50. The measurement calculator 50 includes a Jacobian matrix calculator 3, an electrical property distribution calculator 4, and an outputter 5.

The measurement calculator 50 of the massage apparatus 100 includes, for example, a central processing unit (CPU), a read only memory (ROM), a random access memory (RAM), and a hard disk drive (HDD)/solid state drive (SSD). The Jacobian matrix calculator 3, the electrical property distribution calculator 4, and the outputter 5 are realized by executing a predetermined program in the CPU. The program may be acquired via a recording medium or a network. In addition, a dedicated hardware configuration for realizing a configuration of massage apparatus 100 may be used. The respective elements will be described below.

### Pressing Measurer

The pressing measurer 30 will be described with reference to FIG. 2. FIG. 2 is a schematic diagram of the pressing measurer 30. In the drawings used in the following description, in order to facilitate understanding of features, features may be illustrated in an enlarged manner for convenience, and dimensional ratios and the like of individual components may be different from actual ratios and the like. Materials, dimensions, and the like provided in the following description are merely examples, and the present invention is not limited thereto and can be appropriately modified and implemented within the scope of achieving the effects of the present invention. The pressing measurer 30 includes an in-vivo measurer 1 that measures a change in biological information of a subject due to pressing, a plurality of pressers 20 that press the subject, and a pressing controller 25 that controls magnitudes of pressure of the pressers 20.

First, directions will be defined. Here, a case where the subject stands on a floor F and measurement is performed will be described as an example. One direction parallel to the floor F is defined as an x direction, and a direction which is orthogonal to the x direction and along the floor F is defined as a y direction. A z direction is a direction perpendicular to the floor F. The z direction is a direction orthogonal to the x direction and the y direction. Hereinafter, a +z direction may be referred to as “upward”, and a −z direction may be referred to as “downward”. The upward and downward do not necessarily match the direction in which gravity is applied.

### Presser

The presser 20 is not particularly limited as long as the presser can press the subject, and a known pressing means used for massage can be used. Examples of the presser 20 include an air bag and a massage ball. The air bag is preferably used because appropriate pressure can be applied to the subject by inflating and deflating the air bag. In the present embodiment, the air bag will be described as an example. In the present embodiment, the presser 20 has one air bag 21. Each presser 20 is connected to the pressing controller 25 via a flow channel 22.

The number of pressers 20 is, for example, two or more, and preferably four or more. As the number of pressers 20 increases, it is possible to more finely set pressing regions. Consequently, interstitial fluid, lymphatic fluid, and venous blood can more efficiently recirculate. In the present embodiment, four pressers 20 are provided. The upper limit of the number of pressers 20 is not particularly limited and is, for example, 20.

The presser 20 may have a pressure sensor (not illustrated) for measuring pressure applied to the subject. Since the pressure sensor is provided at the presser 20, a relationship between applied pressure and a change in biological information can be ascertained in more detail.

In the massage apparatus 100 of the first embodiment, the pressers 20 are arranged on a leg of the subject. In the present embodiment, the pressers are arranged to cover the perimeter of the practitioner (the perimeter of the leg). Since the presser 20 has one air bag 21, the perimeter of the leg on which the presser 20 is disposed is pressed with uniform pressure.

In the massage apparatus 100, the plurality of pressers 20 are arranged at intervals in a longitudinal direction of the leg of the subject. Here, the leg refers to a part below the crotch. The leg refers to a part of a human body from the thigh to the ankle. In addition, the longitudinal direction of the leg refers to a direction from the thigh toward the calf when the practitioner stands upright. The plurality of pressers 20 are arranged at intervals in the longitudinal direction of the leg of the subject, and thereby accumulated interstitial fluid, lymphatic fluid, and venous blood can recirculate, for example.

The pressers 20 are preferably arranged between sensors 10 for electrical impedance tomography. By arranging the pressers 20 in this manner, it is possible to ascertain the sensor 10 for electrical impedance tomography toward which the interstitial fluid, the lymphatic fluid, and the venous blood has moved due to pressing.

### Pressing Controller

The pressing controller 25 temporally and spatially controls the pressure of each of the pressers 20 so that the interstitial fluid, the lymphatic fluid, and the venous blood can recirculate. The temporal and spatial application method of the pressure is not particularly limited as long as the interstitial fluid, the lymphatic fluid, and the venous blood can recirculate. The pressing controller 25 controls pumps (not illustrated) that send air to the pressers 20 and solenoid valves (not illustrated) for controlling the amount of air sent into the pressers 20 and controls the pressure of each presser 20.

### In-Vivo Measurer

The in-vivo measurer I includes one or more sensors 10 for electrical impedance tomography and an electric controller 40. A current or a potential difference is applied to the subject by using the sensor 10 for electrical impedance tomography, and thereby internal structures of the four limbs of the subject can be visualized. A temporal change of visualized internal biological information (for example, an electrical property distribution such as a conductivity distribution) is observed, and thereby it is possible to ascertain a change in distribution of interstitial fluid, lymphatic fluid, and venous blood.

It is preferable that the in-vivo measurer 1 include two or more sensors 10 for electrical impedance tomography, because the flow of interstitial fluid, lymphatic fluid, and venous blood can thus be ascertained. As the number of the sensors 10 for electrical impedance tomography increases, the flow of interstitial fluid, lymphatic fluid, and venous blood can be ascertained more accurately.

When the subject is wearing the sensors 10 for electrical impedance tomography, the in-vivo measurer 1 preferably applies a current or a potential difference between electrodes 15, measures a potential difference and a phase based on a current application/voltage measurement pattern to be described below in a case where a current is applied, and measures a current and a phase based on a voltage application/current measurement pattern to be described below in a case where a potential difference is applied between the electrodes 15.

In the case where a current is applied, the in-vivo measurer 1 measures a potential difference based on a predetermined current application/voltage measurement pattern (pattern in which two electrodes are sequentially selected from a large number of electrodes, the current is applied, and the potential difference is sequentially measured). At this time, it is desirable that the in-vivo measurer 1 also measure a phase (a temporal difference between the applied current and the measured potential difference). In the case where a potential difference is applied, the in-vivo measurer I measures a current based on a predetermined voltage application/current measurement pattern (pattern in which two electrodes are sequentially selected from a large number of electrodes, the potential difference is applied, and the current is sequentially measured). At this time, it is preferable that the in-vivo measurer I also measure a phase (a temporal difference between the applied potential difference and the measured current). The following description focuses on the case where a current is applied, and a detailed description of the case where a potential difference is applied may be omitted.

### Sensor for Electrical Impedance Tomography

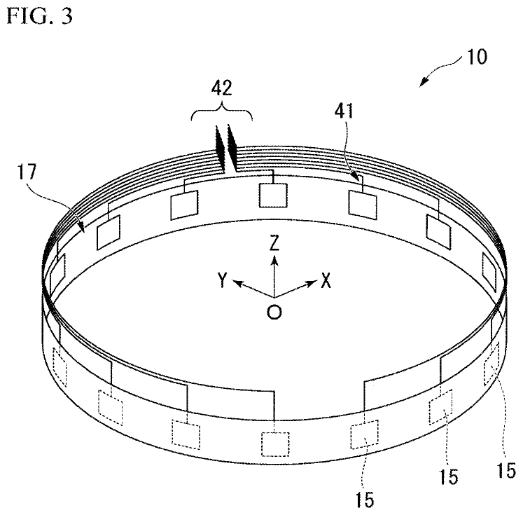

As illustrated in FIG. 3, the sensor 10 for electrical impedance tomography of the first embodiment includes four or more electrodes 15 (the number Q of electrodes) and a support 17.

The electrodes 15 are electrically connected to the electric controller 40. A material and a shape of the electrode 15 are not particularly limited as long as a current or a potential difference can be applied to the subject. Examples of the electrode 15 include a metal such as Au, Ag, or Cu, a conductive polymer, a fiber having a surface coated with a metal, a fiber having a surface coated with a conductive polymer, and the like.

The number Q of the electrodes 15 is four or more. The number of electrodes 15 is four or more, and thereby it is possible to estimate an electrical property distribution that is biological information of the subject by using a calculation result of the Jacobian matrix calculator 3 to be described below. It is preferable that a large number of electrodes be provided in order to enhance the accuracy of calculation.

Arrangement positions of the electrodes 15 are not particularly limited. The electrodes 15 are preferably arranged at uniform intervals to surround the perimeter of the subject (here, the perimeter of the leg).

An electrical connection method between the electrodes 15 and the electric controller 40 is not particularly limited, and a known electrical connection method can be used. In the present embodiment, the electrodes 15 and the electric controller 40 are connected by electric wires 41. Each of the sensors 10 for electrical impedance tomography and the electric controller 40 are connected to an electric wire bundle 42 in which the electric wires 41 are bundled.

The support 17 is not particularly limited as long as the support 17 can hold the electrodes 15. It is preferable that the support 17 enable the electrodes 15 to be arranged in respective regions near pressing scheduled regions of the subject. Here, “capable of being arranged in the regions near the pressing scheduled regions of the subject” means that the electrodes 15 are arranged in the respective regions near the pressing scheduled regions of the subject when the subject is wearing the sensors 10 for electrical impedance tomography. The regions near the pressing scheduled regions are regions adjacent to regions pressed by the pressers 20 and refers to regions where a distribution of interstitial fluid, lymphatic fluid, venous blood, and the like changes due to the pressing. The region near the pressing scheduled region refers to, for example, a region within a range of 0 cm or longer and 10 cm or shorter from the presser 20.

It is preferable that the support 17 can apply a predetermined pressure to such an extent that the electrodes 15 can be brought into close contact with the subject. Consequently, close contact between the electrodes 15 and the subject is enhanced, and a current or a potential difference can be more accurately applied such that the potential difference or the current can be measured. A material of the support 17 is not particularly limited, and for example, it is preferable to use an insulator such as elastomer, leather, and cloth. The shape of the support 17 is not particularly limited, and examples thereof include a boot shape and a band shape.

### Electric Controller

The electric controller 40 includes, for example, a multiplexer for switching between a current application electrode to which a current is applied (or a voltage application electrode to which a potential difference is applied) and a voltage measurement electrode that measures a potential difference (or a current measurement electrode that measures a current), an impedance analyzer that performs voltage measurement (or current measurement) and phase measurement, or the like. The impedance analyzer is a component that changes the applied frequency and the amplitude to measure impedance, that is, the ratio of a measured potential difference (applied potential difference) to the applied current (measured current), and the phase thereof. The electric controller 40. for example, executes a predetermined program in the CPU and controls the multiplexer and the impedance analyzer, thereby performing impedance measurement (measurement of the ratio of the potential difference to the current and the phase thereof). The electric controller 40 may be controlled only inside the in-vivo measurer 1 to perform the impedance measurement, or the electric controller 40 may be controlled according to a program executed by the measurement calculator 50 to perform the impedance measurement. A result of the impedance measurement is sent to the electrical property distribution calculator 4. A method for transmitting information to the electrical property distribution calculator 4 is not particularly limited. The information may be sent from the electric controller 40 to the electrical property distribution calculator 4 of the measurement calculator 50 in a wired manner or may be sent to the electrical property distribution calculator 4 of the measurement calculator 50 in a wireless manner.

The electric controller 40 applies a current between the electrodes 15 based on the predetermined current application/voltage measurement pattern (pattern in which a current is applied to certain electrodes and a potential difference is applied between certain electrodes), and measures the potential difference. Alternatively, the electric controller 40 applies the potential difference between the electrodes 15 based on the predetermined voltage application/current measurement pattern and measures the current. Similarly in the case where a current is applied and also in the case where a potential difference is applied, certain electrodes 15 between which a current (potential difference) is applied and certain electrodes between which the potential difference (current) is measured are not particularly limited, but it is preferable to apply the current (potential difference) “evenly” to the arranged electrodes 15 and measure the potential difference (current). “Applying the current (potential difference) evenly and measuring the potential difference (current)” means applying and measuring the current potential difference such that all the electrodes 15 are used for applying or measuring the current potential difference once. Note that the current application/voltage measurement pattern to be described below can also be applied to the voltage application/current measurement pattern. The current value to be applied and the application frequency thereof are preferably, for example, AC from a Hz band of 1.0 mA or lower to about a MHz band in view of an effect on a living body or convenience of the apparatus.

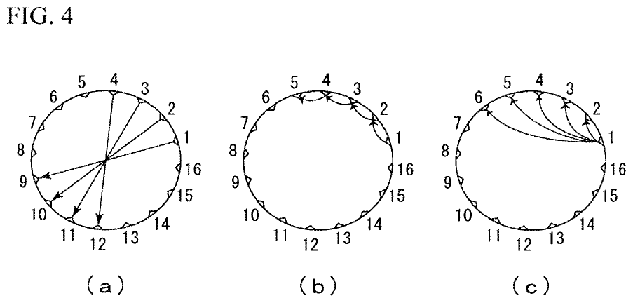

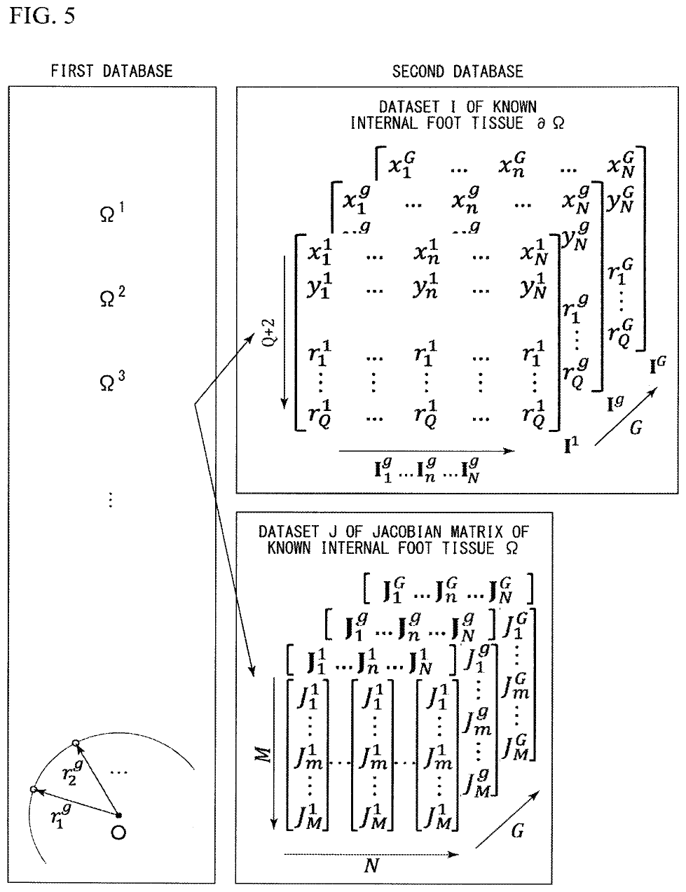

A current application/voltage measurement pattern to the electrodes 15 will be described using an electrode arrangement of FIG. 4 as an example. Numbers representing the positions of the electrodes 15 are numbered, for example, counterclockwise from a first electrode serving as a reference. The number M of current application/voltage measurement patterns is different for each current application/voltage measurement pattern. Hereinafter, each current application/voltage measurement pattern will be described. Hereinafter, examples of the current application/voltage measurement patterns will be described, but the present invention is not limited to the following current application/voltage measurement patterns.

First, current application/voltage measurement patterns by a counter-polar method will be described. In this case, a current is applied between a pair of opposing electrodes. For example, with reference to FIG. 4(a), a current is applied to opposing electrodes such as the first electrode and the ninth electrode, and the second electrode and the tenth electrode. In the case of FIG. 4(a), since the number Q of electrodes is 16, there are eight combinations in total. A potential difference is measured from electrode pairs such as the second electrode and the third electrode, and the third electrode and the fourth electrode excluding the electrodes to which the current is applied, and is measured from the electrode pair of the second electrode and the third electrode to an electrode pair of the fifteenth electrode and the sixteenth electrode, so that there are 13 voltage measurement patterns for one current application pattern. Hence, in the case of the counter-polar method, the number of measurements (measurement patterns) M is 104 in total. Here, in a case where a current is applied and a potential difference is measured, a measurement pattern thereof is a voltage measurement pattern. In a case where a potential difference is applied and a current is measured, the measurement pattern thereof is a current measurement pattern.

Next, current application/voltage measurement patterns by an adjacent method will be described. In this case, a current is applied between adjacent electrodes. For example, with reference to FIG. 4(b), a current is applied to adjacent electrodes such as the first electrode and the second electrode, and the second electrode and the third electrode. In the case of FIG. 4(b), since the number Q of electrodes is 16, there are 16 combinations in total. The potential difference is measured from an electrode pair such as the third electrode and the fourth electrode excluding the electrodes to which the current is applied, and is measured from the third electrode and the fourth electrode to the fifteenth electrode and the sixteenth electrode, so that there are 13 voltage measurement patterns for one current application pattern. Hence, in the case of the adjacent method. the number of measurements (measurement patterns) M is 208 in total.

Current application/voltage measurement patterns by a reference method will be described. In this case, a potential difference is measured in all combinations between a reference electrode and electrodes other than the reference electrode. For example, with reference to FIG. 4(c), a current is applied between a reference electrode and an electrode other than the reference electrode, such as the first electrode and the second electrode, and the first electrode and the third electrode. In the case of FIG. 4(c), since the number Q of electrodes is 16, there are 16 combinations in total. The potential difference is measured from electrode pairs such as the third electrode and the fourth electrode excluding the electrodes to which the current is applied, and is measured from the electrode pair of the third electrode and the fourth electrode to an electrode pair of the fifteenth electrode and the sixteenth electrode, so that there are 13 voltage measurement patterns for one current application pattern. Hence, in the reference method, the number of measurements (measurement patterns) M is 208 in total.

Hereinafter, an example in which the potential difference is measured using the adjacent method in the massage apparatus 100 of the present embodiment will be described. Note that, in the following description, a calculation example for one sensor 10 for electrical impedance tomography will be described, but calculation can be similarly performed in a case where there are two or more sensors 10 for electrical impedance tomography.

### Jacobian Matrix Calculator 3

The Jacobian matrix is a sensitivity matrix indicating how much a measured potential difference when a current is applied (or a measured current when voltage is applied) changes with respect to a change in electrical properties (conductivity or permittivity) distributed in a space with respect to a reference. The Jacobian matrix (sensitivity matrix) of the subject varies depending on a spatial distribution, a body shape, or the like of the electrical properties of the subject, and if the Jacobian matrix of the subject is known, an electrical property distribution can be calculated. The Jacobian matrix calculator 3 uses predetermined current application/voltage measurement patterns (or voltage application/current measurement patterns), mesh coordinates obtained by dividing a contour of the subject measured in advance and coordinates of the electrodes 15 to calculate a Jacobian matrix J* of an internal structure Ω of the subject (* is a symbol meaning that estimation has been performed for the subject). In the present embodiment, the mesh coordinates obtained by dividing the contour of the subject indicate a leg contour of the subject (mesh coordinates obtained by dividing the leg contour ∂Ω). The Jacobian matrix calculator 3 may (1) calculate the Jacobian matrix J* by using the following Expression (9) on the basis of an X-ray image, an MRI image, or the like of the subject's own internal leg tissue imaged in advance and create the Jacobian matrix J* on a tailor-made basis (tailor-made Jacobian matrix J*), and (2) set, as a first database, three-dimensional position information of fat, muscles, and bones in the leg and all shapes of G leg contours ∂Ω based on general information such as age, gender, nationality, height, and weight, (3) create a second database (a data set I of known leg contours ∂Ω and a data set J of the Jacobian matrix of the known internal leg tissue Ω) from the first database, and, (4) select an optimal Jacobian matrix J* of the subject by using machine learning or the like for the leg contours ∂Ω of the subject from the second database. Here, the leg contours ∂Ω of the subject refer to leg contours of the subject in the regions where the electrodes 15 are arranged. In the present embodiment, the case of a leg will be described as an example, but the present invention can also be applied to an arm. an abdomen, or the like. In the case of application to the arm, contours of the arm are used, and in the case of application to the abdomen, contours of the abdomen are used.

Hereinafter, the first database and the second database in (2) will be described with reference to FIG. 5. Here, the leg of the subject will be described as an example, but the present invention can be applied not only to the leg but also to an arm, an abdomen, or the like. First, in the first database, for example, information of the G leg contours ∂Ω of all shapes (geometric shapes) on the assumption of a fat person or a thin person (for example, an image obtained by processing a generally available 3D image on the assumption of a fat person or a thin person) is prepared for generally available leg contour information of a healthy person having a specific age, nationality, or gender (for example, a generally available 3D image of a leg).

In the first database, for a leg contour ∂Ω ^{g} of the g-th geometric shape, an internal structure Ω^{g} thereof is divided into two-dimensional meshes to obtain an appropriate resolution according to Q electrodes 15. For example, in a case where the number Q of electrodes 15 is 16, a region including the known leg contour ∂Ω^{g} may be divided into a total of 4,096 points of 64 points in the x direction and 64 points in the y direction to create meshes n (1≤n≤N). In this case, N is 4096. The number and the shape of the meshes can be appropriately set according to the number of electrodes 15 and required resolution. Note that this work may be performed in the first database or in the next second database.

Next, the data set I of the known leg contours ∂Ω for the information of the G leg contours ∂Ω of the geometric shapes in the second database will be described. The data set I of the known leg contours ∂Ω is a matrix including data of the known leg contours ∂Ω having elements of (Q+2)×N (the number of spatial meshes)×G (the number of geometric shapes in the first database). Here, Q precisely represents the number of contour measurement points and may take a value different from the number of electrodes, but here, for convenience, the number Q of contour measurement points and the number Q of electrodes are the same value. The data set I of the leg contours ∂Ω includes the measured contour ∂Ω of the subject, N mesh coordinates (xₙ, yₙ) obtained by dividing the internal leg tissue Ω from the leg contour ∂Ω, and a distance r from the origin O to each electrode 15. The meaning of 2 in Q +2 is a coordinate position (xₙ, yₙ) in an n-th mesh of a g-th geometric shape, and the meaning of Q is a radius r of the contour measurement point Q in the g-th geometric shape.

The data set I is represented by the following Expression (1). Here, I^{g} denotes an input variable in the known leg contour ∂Ω^{g} and is represented by the following Expression (2). In Expression (2), I^{g}ₙ denotes an input variable of the mesh n in the known leg contour ∂Ω^{g} and is represented by the following Expression (3). In Expression (3), X^{g}ₙ, denotes a Cartesian coordinate (xₙ, yₙ) of the meshes n in the known leg contour ∂Ω^{g} and is represented by the following Expression (4). In Expression (3), r^{g} denotes a distance from the origin of the electrode 15 disposed at the known leg contour ∂Ω^{g} and is represented by the following Expression (5). In Expression (5), Q denotes the number of contour measurement points (which may be the same as the number of electrodes). Note that T in Equations denotes transposition of matrix elements. R on the right side denotes a set of real numbers, a superscript thereof denotes an element of a matrix or an element of a column vector, and a subscript thereof indicates an element of a row vector.

$$\begin{matrix}[ Math . 1 ] & \\ I = [ {I}^{1} , ... , {I}^{g} , ... , {I}^{G} ] ∈ {( Q + 2 ) \times N \times G}^{} & ( 1 )\end{matrix}$$

$${I}^{g} = [ {{I}^{g}}_{1} , ... , {{I}^{g}}_{n} , ... , {{I}^{g}}_{N} ] ∈ {( Q + 2 ) \times N}^{} \qquad ( 2 )$$

$${{I}^{g}}_{n} = {[ {{X}^{g}}_{n} , {r}^{g} ]}^{T} ∈ {( Q + 2 )}^{} \qquad ( 3 )$$

$${X}_{n}^{g} = [ {x}_{n} , {y}_{n} ] ∈ {2}_{} \qquad ( 4 )$$

$${r}^{g} = [ {r}_{1}^{g} , ... , {r}_{q}^{g} , ... , {r}_{Q}^{g} ] ∈ {Q}_{} \qquad ( 5 )$$

Regarding the data set J of the Jacobian matrix of the known internal leg tissue Ω, for example, the data set J of the known Jacobian matrix may be created by calculating a Jacobian matrix J by using a finite element method from the first database described above and the leg contours ∂Ω of all shapes. Since the Jacobian matrix J varies depending on the leg contours ∂Ω of the subject, it is preferable to prepare a large number of known leg contours ∂Ω and the Jacobian matrix J. Here, the known leg contour ∂Ω is set to ∂Ω^{g} (1≤g≤G). G is the number of known leg contours ∂Ω in the dataset and is, for example, 100 to 10,000. The type of ∂Ω^{g} and the number of G can be appropriately corrected. The data set J of the Jacobian matrix of the known internal leg tissue Ω is a known sensitivity matrix having elements of M (the number of current application/voltage measurement patterns)×N (the number of spatial meshes)×G (the number of first databases). G two-dimensional shapes are X vectors and refer to data of a plurality of Jacobian matrices J acquired from leg contours ∂Ω of a plurality of known samples by the finite element method or the like. The data set J of the Jacobian matrix is a sensitivity matrix of the predetermined current application/voltage measurement patterns (or the voltage application/current measurement patterns).

The Jacobian matrix J of the leg contours ∂Ω is represented by the following Expression (6). In Expression (6), M denotes the number of current application/voltage measurement patterns, N denotes the number of meshes, and G denotes the number of geometric shapes. A Jacobian matrix J^{g} of a geometric shape g (1≤g≤G) is represented by Expression (7), and a Jacobian matrix J^{g}ₙ in the meshes n (1 <n <N) of the geometric shape g (1≤g≤G) is represented by Expression (8). A Jacobian matrix element J^{g}ₙₘ in a current application/voltage measurement pattern m (1≤m≤M) of the meshes n (1≤n<N) of the geometric shape g (1≤g≤G) is calculated using the following Expression (9). Here, σₙ denotes the conductivity in the meshes n as an example of an electrical property distribution, but the conductivity may be an example of another electrical property distribution (a conductivity difference distribution Δσ, a permittivity distribution, a permittivity difference distribution, a phase distribution, or a phase difference distribution). Aₙ represents an n-th mesh area, but in a case where it is desired to perform simple calculation due to calculation costs or the like, a mesh area in an x-y direction may be used to approximate an area in the z direction. Vm (e, d) denotes a measured potential difference V in the current application/voltage measurement pattern m. In addition, e denotes a current application electrode pair in the current application/voltage measurement pattern m, and d denotes a voltage measurement electrode pair in the current application/voltage measurement pattern m. V(i^{e}) denotes a potential difference between a voltage measurement electrode pair d induced by current application to a current application electrode pair e. V(i^{d}) denotes a potential difference between the current application electrode pair e induced by current application to the voltage measurement electrode pair d. ∇ denotes a nubbler symbol and denotes a differential operator.

$$\begin{matrix}[ Math . 2 ] & \\ J = [ {J}^{1} , ... , {J}^{g} , ... , {J}^{G} ] ∈ {M \times N \times G}^{} & ( 6 )\end{matrix}$$

$${J}^{g} = [ {{J}^{g}}_{1} , ... , {{J}^{g}}_{n} , ... , {{J}^{g}}_{N} ] ∈ {M \times N}^{} \qquad ( 7 )$$

$${{J}^{g}}_{n} = {[ {{J}^{g}}_{n 1} , ... , {{J}^{g}}_{nm} , ... , {{J}^{g}}_{nM} ]}^{T} ∈ {M}^{} \qquad ( 8 )$$

$${J}_{nm}^{g} = \frac{\partial {V}_{m} ( e , d )}{\partial {σ}_{n}} = - {\int }_{{A}_{n}} ∇ V ( {i}^{e} ) \cdot ∇ V ( {i}^{d} ) {dA}_{n} \qquad ( 9 )$$

Hereinafter, a method of calculating the Jacobian matrix J* of the subject (* is a symbol meaning that estimation has been performed for the subject) from the leg contours ∂Ω of the subject based on the predetermined current application/voltage measurement pattern m (1≤m≤M) by using the data set J of the Jacobian matrix of the known internal leg tissue Ω and the data set of the known leg contours ∂Ω output from the second database of FIG. 5 will be described. When the Jacobian matrix J* is calculated, the data set I of the leg contours ∂Ω and the data set J of the Jacobian matrix of the internal leg tissue Ω are used as input variables, and the Jacobian matrix J* of the subject is calculated using, for example, a nearest neighbor search technique or machine learning such as a neural network. The nearest neighbor search technique is not particularly limited, and examples thereof include a K-nearest neighbor algorithm, an approximate nearest neighbor search, locality sensitive hashing, and a k-d tree. The case of using the neural network corresponds to, for example, the above-described “tailor-made Jacobian matrix J*”. That is, spatial position information of tissue such as fat, muscle, and bone is already known from an X-ray image or an MRI image of the subject's own internal leg tissue imaged in advance, and spatial position information of conductivity and permittivity is also already known. In a known object, a voltage value when the current is applied between the electrodes is already known by actually measuring the subject himself/herself or known by using electromagnetic calculation or the like without actually measuring the voltage value. That is, since the Jacobian matrix J* is a physical quantity connecting the spatial position information of the conductivity or the permittivity which is an input value and the spatial position information of the tissue which is an output value, J* can be obtained by using the neural network even if the relationship between the two items of information is not strongly formulated as nonlinear.

Hereinafter, the K-nearest neighbor algorithm will be described as an example.

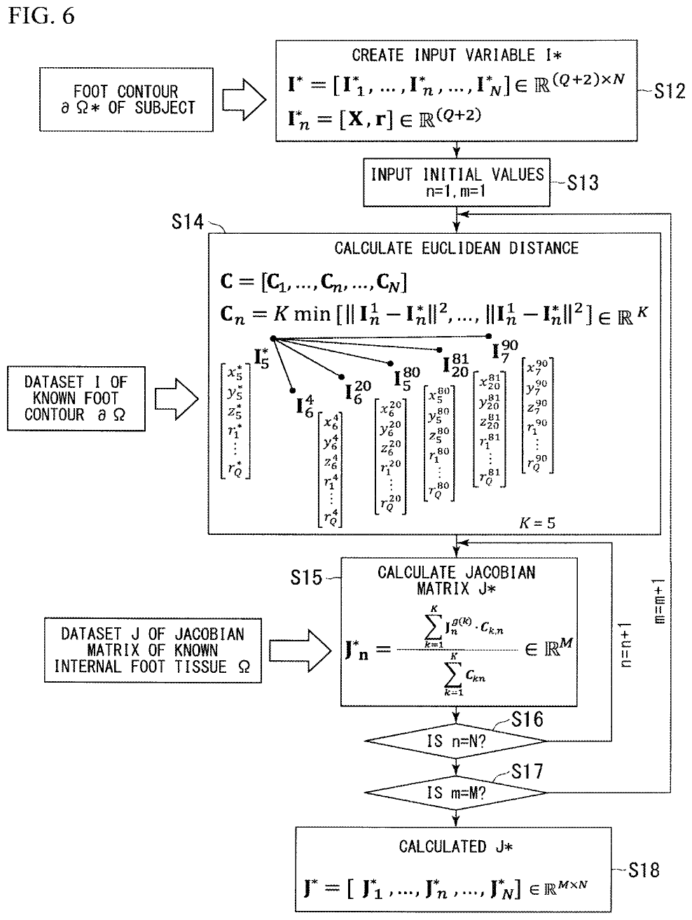

Next, a method of calculating the Jacobian matrix J* of the subject by the K-nearest neighbor algorithm will be described with reference to FIG. 6. The Jacobian matrix calculator 3 divides a leg contour ∂Ω* of the subject to have the same number N of meshes as that of a data set of the known leg contour ∂Ω^{g}. Thereafter, an input variable I* is created from the meshes n from the predetermined current application/voltage measurement pattern m and the coordinates of the electrodes 15 (S12). The input variable I* denotes an input variable in the leg contour ∂Ω* of the subject and is represented by the following Expression (10). In Expression (10), I*ₙ denotes an input variable of the meshes n in the leg contours ∂Ω of the subject and is represented by the following Expression (11). In Expression (11), X*ₙ denotes Cartesian coordinates (x*ₙ, y*ₙ) of the meshes n in the leg contours ∂Ω of the subject and is represented by the following Expression (12). T denotes transposition of the matrix elements. In Expression (11), r* denotes a distance from the origin O of each electrode 15 (precisely, a contour measurement point) arranged at the leg contours ∂Ω of the subject and is represented by the following Expression (13). In Expression (13), Q denotes the number of the electrodes 15 (precisely, the contour measurement points).

$$\begin{matrix}[ Math . 3 ] & \\ {I}^{*} = [ {{I}^{*}}_{1} , ... , {{I}^{*}}_{n} , ... , {{I}^{*}}_{N} ] ∈ {( Q + 2 ) \times N}^{} & ( 10 )\end{matrix}$$

$${{I}^{*}}_{n} = {[ {X}_{n}^{*} , {r}^{*} ]}^{T} ∈ {( Q + 2 )}^{} \qquad ( 11 )$$

$${X}_{n}^{*} = [ {x}_{n}^{*} , {y}_{n}^{*} ] ∈ {2}_{} \qquad ( 12 )$$

$${r}^{*} = [ {r}_{1}^{*} , ... , {r}_{q}^{*} , ... , {r}_{Q}^{*} ] ∈ {Q}_{} \qquad ( 13 )$$

Next, flow of calculation of the Jacobian matrix J* of the subject by the K-nearest neighbor algorithm (K-NN) will be described. The Jacobian matrix calculator 3 inputs initial values (for example, n=1, and m=1) (S13). Next, the Jacobian matrix calculator 3 first calculates a Euclidean distance matrix C between the measured I* of the subject and the data set I of the known leg contours ∂Ω which is an output from the second database (S14). The Euclidean distance matrix C is represented by the following Expression (14) and indicates an input variable I^{g}ₙₘ of K clusters having a small Euclidean distance from I*ₙₘ. The number K of clusters is not particularly limited, and is, for example, five. The Euclidean distance matrix C includes elements K×N of the number K of clusters and the number N of meshes which are independently determined. A Euclidean distance matrix Cn in the meshes n (1≤n≤N) is represented by the following Expression (15), indicates a Euclidean distance between I^{g}ₙ and I*ₙ, and is determined such that a data set I^{g} of the measured I* of the subject and the known leg contours ∂Ω of the geometric shape g (1≤g≤G) is minimized. Next, a Jacobian matrix J*ₙ (the number of elements is M) of the subject in m when n is fixed is calculated using the Euclidean distance matrix C and the data set J of the Jacobian matrix of the known internal leg tissue Ω (S15). This is calculated by the following Expression (16), and J^{g}ₙ denotes a Jacobian matrix of the current application/voltage measurement pattern m in meshes n in the known leg contour ∂Ω^{g}. In FIG. 6, for example, an example of I₅* is described using the position of the meshes n=5 as an example.

When the calculation of Jₙₘ* is completed, the Jacobian matrix calculator 3 determines whether the number of n is equal to the number N of meshes (S16). In a case where n and N are not equal to each other, the number of n is increased by one, and this processing returns to S15 again (S16). In the case where n and N are equal to each other, next, it is determined whether m is equal to the number M of the current application/voltage measurement patterns (S17). In a case where m and M are not equal to each other. the number of m is increased by one, and this processing returns to S14 again (S17). When n and m become equal to N and M, respectively, the Jacobian matrix calculator 3 ends the calculation of the Jacobian matrix J* of the subject (S18) and sends the Jacobian matrix J* of the subject to the electrical property distribution calculator 4. If the Jacobian matrix J is calculated by a normal personal computer using the above Expression (9) without using machine learning, the calculation is performed in 5 minutes or longer. By preparing the known data set I and the data set J of the Jacobian matrix of the leg contour ∂Ω^{g} and using machine learning such as the K-nearest neighbor algorithm based on the leg contours ∂Ω of the subject and the current application/voltage measurement pattern, the Jacobian matrix J* of the subject can be calculated with high accuracy in a short time.

$$\begin{matrix}[ Math . 4 ] & \\ C = [ {C}_{1} , ... , {C}_{n} , ... , {C}_{N} ] ∈ {K \times N}^{} & ( 14 )\end{matrix}$$

$${C}_{n} = K min [ {\sum }_{g = 1}^{g = G} {\sum }_{n = 1}^{N} ‖ {I}_{n}^{*} - {I}_{n}^{g} ‖ ] ∈ {K}^{} \qquad ( 15 )$$

$${{J}^{*}}_{n} = \frac{{\sum }_{k = 1}^{K} {J}_{n}^{g ( k )} {C}_{n , k}}{{\sum }_{k = 1}^{K} {C}_{n , k}} ∈ {M}^{} \qquad ( 16 )$$

### Electrical Property Distribution Calculator

The electrical property distribution calculator 4 calculates electrical property distributions of the subject from the Jacobian matrix J #of the subject sent from the Jacobian matrix calculator 3 and the potential difference and the phase (or the current and the phase) measured by the in-vivo measurer 1. Here, the electrical property distributions are, for example, a conductivity distribution σ, a conductivity difference distribution Δσ, a permittivity distribution, a permittivity difference distribution, a phase distribution, and a phase difference distribution. Hereinafter, the conductivity and the conductivity difference (the conductivity at a time t with respect to a reference at a time t₀) may be distinguished and described, and Δ is used as a symbol denoting a difference.

The following description focuses on the conductivity difference distribution Δσ. A problem to obtain the conductivity difference distribution Δσ from the known Jacobian matrix Je of the subject and a known measured potential difference ΔV (potential difference at the time t with respect to the reference of the time t₀) is referred to as an inappropriate inverse problem and can be solved using, for example, repeated calculations. The number of repetitions is denoted by a number on the right side. An initial conductivity difference distribution Δσ⁰ at the 0-th repetition count (the upper right number denotes the number of repetitions) is calculated from the following Expression (17) using the Jacobian matrix J* of the subject. T denotes a transposed matrix. In Expression (17), ΔV denotes a column vector having M elements of a predetermined current application/voltage measurement pattern (or voltage application/current measurement pattern) as represented by Expression (18) below. There are two ways of processing ΔVm in Expression (18), that is, a method of using a measurement time difference at a constant application current frequency and a method of using several application current frequency differences at a constant measurement time. Here, in the description of the method of using the measurement time difference, the method is represented by the following Expression (19) using the measurement potential difference ΔV of a time difference from a measurement potential ΔVₘ(t) at the time t with a measurement potential ΔVₘ(t₀) at the time to of the current application/voltage measurement pattern m (0≤m≤M) as a reference. In addition, this expression may be divided by Vₘ(t₀). In Expression (19), m denotes the current application/voltage measurement pattern.

The conductivity difference distribution Δσ of the subject is calculated using the following Expression (20) with the initial conductivity difference distribution Δσ⁰ as a start of the number of repetitions. In Expression (20), i denotes the number of repeated calculations. In Expression (20), R denotes a regularization matrix, and λ denotes an arbitrary parameter for converging the calculations and is set to, for example, 0.01. R is represented by the following Expression (21), for example, and is a function of the known Jacobian matrix J* of the subject. The calculated electrical property distribution (here, the conductivity difference distribution Δσ) of the subject is sent to the outputter 5.

$$\begin{matrix}[ Math . 5 ] & \\ {Δσ}^{0} = {J}^{* T} Δ V & ( 17 )\end{matrix}$$

$$Δ V = {[ Δ {V}_{1} , ... , Δ {V}_{m} , ... , Δ {V}_{M} ]}^{T} ∈ {M}^{} \qquad ( 18 )$$

$$Δ {V}_{m} = {V}_{m} ( t ) - {V}_{m} ( {t}_{0} ) \qquad ( 19 )$$

$${Δσ}^{i + 1} = {Δσ}^{i} - {( {J}^{* T} {J}^{*} + λ R )}^{- 1} {J}^{* T} Δ V \qquad ( 20 )$$

$$R = diag ( {J}^{* T} {J}^{*} ) \qquad ( 21 )$$

### Outputter

The outputter 5 outputs the electrical property distributions such as the conductivity distribution σ, the conductivity difference distribution Δσ, the permittivity distribution, the permittivity difference distribution, the phase distribution, and the phase difference distribution calculated by the electrical property distribution calculator 4. These electrical property distributions may be converted into a three-dimensional (3D) image of a two-dimensional space and a time, a 3D image with a fixed time, a one-dimensional (1D) value obtained by spatial mean normalization of the image of the two-dimensional space, a time average value obtained by temporally averaging the image, or the like, and outputs thereof may be displayed. In addition, a two-dimensional image or the like may be displayed for each of the sensors 10 for electrical impedance tomography. An output destination of the outputter 5 is not particularly limited. The output destination may be a display such as a liquid crystal display or a storage device such as an HDD. Hereinafter, <> may be used as a symbol of a spatial mean.

The massage apparatus 100 according to the present embodiment has been described in detail above. Since the massage apparatus 100 can ascertain the change in the biological information inside the practitioner (change in the electrical property distribution), the subject can ascertain whether or not the interstitial fluid, the lymphatic fluid, and the venous blood have been sufficiently recirculated. In addition, since the massage apparatus 100 includes two or more sensors 10 for electrical impedance tomography, it is possible to ascertain where the interstitial fluid, the lymphatic fluid, and the venous blood have flowed due to the pressing.

In the first embodiment, the air bag is used for the presser 20, but the presser 20 may be a massage ball. In the first embodiment, since the air bag is used, the flow channel 22 is used. However, when the presser 20 can be driven by electricity, an electric wire may be used instead of the flow channel 22. In the first embodiment, the three sensors 10 for electrical impedance tomography are provided, but one sensor for electrical impedance tomography may be provided. Even if one sensors 10 for electrical impedance tomography is provided, it is possible to measure a temporal change in biological information due to the pressing. The massage apparatus 100 according to the present embodiment is worn over the leg, but may be worn over the arm, the abdomen, or the like. In this case, the contour used for calculation of the Jacobian matrix is a contour of the arm, a contour of the abdomen, and the like.

### Second Embodiment

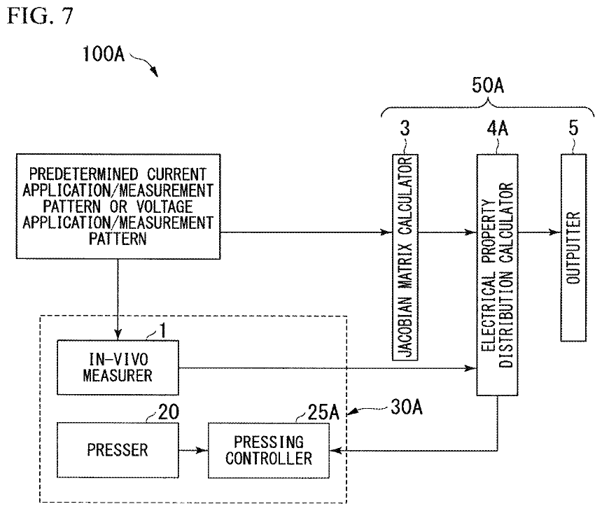

Next, a second embodiment will be described. As illustrated in FIG. 7, a massage apparatus 100A according to the second embodiment includes a pressing measurer 30A and a measurement calculator 50A. The measurement calculator 50A includes a Jacobian matrix calculator 3, an electrical property distribution calculator 4A, and an outputter 5.

Note that, in the second embodiment, the same components as those in the first embodiment are denoted by the same reference numerals, the description thereof is omitted, and only differences will be described.

### Pressing Measurer

The pressing measurer 30A includes an in-vivo measurer 1, a plurality of pressers 20, and a pressing controller 25A.

### Pressing Controller

The pressing controller 25A temporally and spatially controls the pressure of each of the pressers 20 so that the interstitial fluid, the lymphatic fluid, and the venous blood can recirculate. The pressing controller 25A controls the pressure of the presser 20 on the basis of the electrical property distribution of the subject sent from the electrical property distribution calculator 4A of the measurement calculator 50A. For example, in a case where a temporal change of the electrical property distribution obtained by a sensor 10 for electrical impedance tomography is small (in a case of insufficient recirculation of interstitial fluid, lymphatic fluid, and venous blood), the pressing controller controls the pressure of the presser 20 adjacent to the sensor 10 for electrical impedance tomography which obtains the electrical property distribution with a small temporal change to perform pressing so that the interstitial fluid, the lymphatic fluid, and the venous blood can recirculate. As described above, by controlling the presser 20 on the basis of the electrical property distribution, the interstitial fluid, the lymphatic fluid, and the venous blood can recirculate in a shorter time. The pressing controller 25A controls pumps (not illustrated) that sends air to the pressers 20 and solenoid valves (not illustrated) for controlling the amount of air sent into the pressers 20 and controls the pressure of each presser 20.

### Electrical Property Distribution Calculator

The electrical property distribution calculator 4A calculates electrical property distributions of the subject from the Jacobian matrix J* of the subject sent from the Jacobian matrix calculator 3 and the potential difference and the phase (or the current and the phase) measured by the in-vivo measurer 1. The electrical property distribution calculator 4A calculates the electrical property distribution of the subject in the same manner as the electrical property distribution calculator 4. The obtained electrical property distribution of the subject is sent to the outputter 5 and the pressing controller 25A.

The second embodiment has been described above. In the massage apparatus 100A of the second embodiment, the pressing controller 25A adjusts the pressure of the presser 20 according to the electrical property distribution of the subject obtained by the electrical property distribution calculator 4A, and thus the massage can be completed in a shorter time than usual.

### Third Embodiment

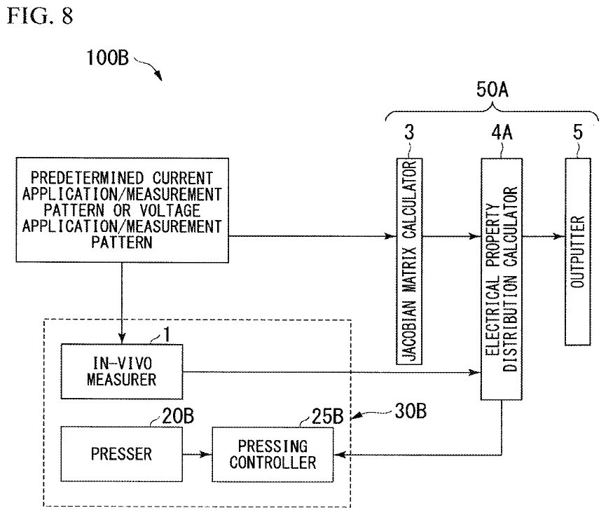

Next, a third embodiment will be described. As illustrated in FIG. 8, a massage apparatus 100B according to the third embodiment includes a pressing measurer 30B and a measurement calculator 50A. The measurement calculator 50A includes a Jacobian matrix calculator 3, an electrical property distribution calculator 4A, and an outputter 5.

Note that, in the third embodiment, the same components as those in the first embodiment and the second embodiment are denoted by the same reference numerals, the description thereof is omitted, and only differences will be described.

### Pressing Measurer

The pressing measurer 30B includes an in-vivo measurer 1, a plurality of pressers 20B, and a pressing controller 25B.

### Presser

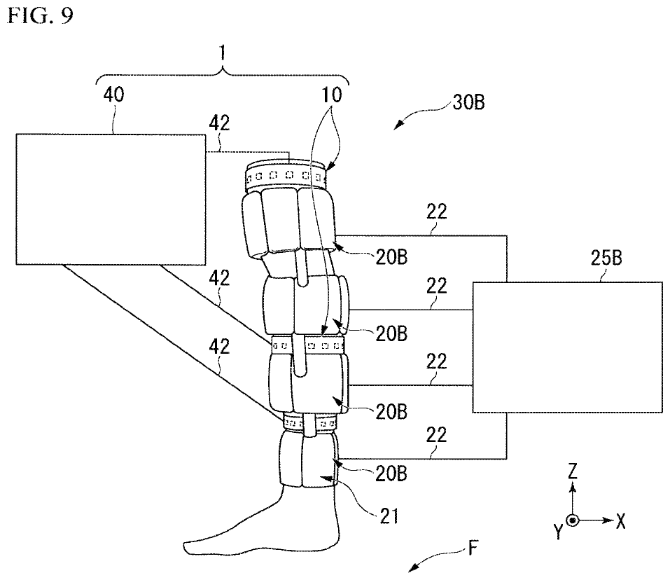

The presser 20B is not particularly limited as long as the presser can press the subject, and a known pressing means used for massage can be used. Examples of the presser 20B include an air bag and a massage ball. The air bag is preferably used because appropriate pressure can be applied to the subject by inflating and deflating the air bag. In the third embodiment, the air bag will be described as an example. As illustrated in FIG. 9, in the present embodiment, the presser 20B has two or more air bags 21. The number of the air bags 21 is preferably three or more, and more preferably four or more. The air bags 21 can apply respective different magnitudes of pressure. As the number of the air bags 21 constituting the presser increases, pressed regions can be finely controlled, and thus an increase in the number of air bags is preferable. The air bag 21 is disposed in a circumferential direction (for example, a circumferential direction of a leg) of the subject. Each air bag 21 of the presser 20B is connected to the pressing controller 25B via a flow channel 22.

The number of pressers 20B is, for example, two or more, and preferably four or more. As the number of pressers 20B increases, it is possible to finely set pressing regions. Consequently, interstitial fluid, lymphatic fluid, and venous blood can more efficiently recirculate. In the present embodiment, four pressers 20B are provided. The upper limit of the number of pressers 20B is not particularly limited and is, for example, 20.

The presser 20B may have a pressure sensor (not illustrated) for measuring pressure applied to the subject. In the present embodiment, since the pressure sensor is provided for each air bag 21 included in the presser 20B, a relationship between applied pressure and a change in biological information can be ascertained in more detail.

In the massage apparatus 100B of the third embodiment, the pressers 20B are arranged to cover the perimeter of the leg of the subject. That is, the plurality of air bags 21 are arranged in the circumferential direction of the leg. Since the plurality of air bags 21 are arranged in the circumferential direction of the leg, the pressure of the air bags 21 can be changed in the circumferential direction to press the subject. Consequently, the accumulated interstitial fluid, lymphatic fluid, and venous blood can more efficiently recirculate.

In the massage apparatus 100B, the plurality of pressers 20B are arranged at intervals in a longitudinal direction of the leg of the subject. The plurality of pressers 20B are arranged at intervals in the longitudinal direction of the leg of the subject, and thereby recirculating the accumulated interstitial fluid, lymphatic fluid, and venous blood in a standing posture, for example.

The pressers 20B are preferably arranged between sensors 10 for electrical impedance tomography. By arranging the pressers 20 in this manner, it is possible to ascertain the sensor 10 for electrical impedance tomography toward which the interstitial fluid, the lymphatic fluid, and the venous blood has moved due to pressing.

### Pressing Controller

The pressing controller 25B temporally and spatially controls the pressure of each of the pressers 20B so that the interstitial fluid, the lymphatic fluid, and the venous blood can recirculate. The pressing controller 25B controls the pressure of the presser 20B on the basis of the electrical property distribution of the subject sent from the electrical property distribution calculator 4A of the measurement calculator 50A. In a case where a change of the electrical property distribution obtained by each of the sensors 10 for electrical impedance tomography is small and the interstitial fluid, the lymphatic fluid, and the venous blood insufficiently recirculate, the pressing controller controls the pressure of each of the air bags 21 in the pressers 20B adjacent to the sensors 10 for electrical impedance tomography which obtain the electrical property distributions with a small temporal change to perform pressing so that the interstitial fluid, the lymphatic fluid, and the venous blood can recirculate. As described above, by controlling each of the air bags 21 in the pressers 20B on the basis of the electrical property distributions, the interstitial fluid, the lymphatic fluid, and the venous blood can recirculate in a shorter time than in the case of the second embodiment. The pressing controller 25B controls pumps (not illustrated) that sends air to the pressers 20 and solenoid valves (not illustrated) for controlling the amount of air sent into the pressers 20 and controls pressure of each of the air bags 21 in the pressers 20B.

The third embodiment has been described above. In the massage apparatus 100B of the third embodiment, the pressing controller 25B adjusts the pressure of each of the air bags 21 of the pressers 20B according to the electrical property distribution of the subject obtained by the electrical property distribution calculator 4A, and thus the massage can be effectively completed in a shorter time.

### Fourth Embodiment

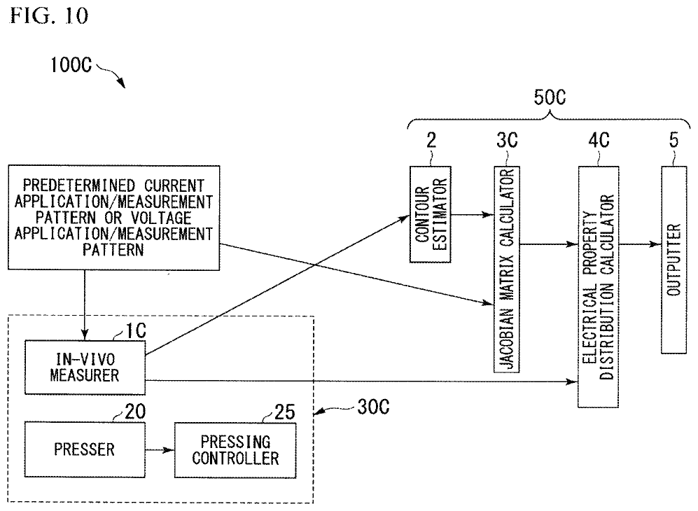

Next, a fourth embodiment will be described. As illustrated in FIG. 10, a massage apparatus 100C according to the fourth embodiment includes a pressing measurer 30C and a measurement calculator 50C. The measurement calculator 50C includes a contour estimator 2, a Jacobian matrix calculator 3C, an electrical property distribution calculator 4C, and an outputter 5. Note that, in the fourth embodiment, the same components as those in the first embodiment, the second embodiment, and the third embodiment are denoted by the same reference numerals, a description thereof is omitted, and only differences will be described.

### Pressing Measurer

The pressing measurer 30C includes an in-vivo measurer 1C, a plurality of pressers 20, and a pressing controller 25.

### In-Vivo Measurer

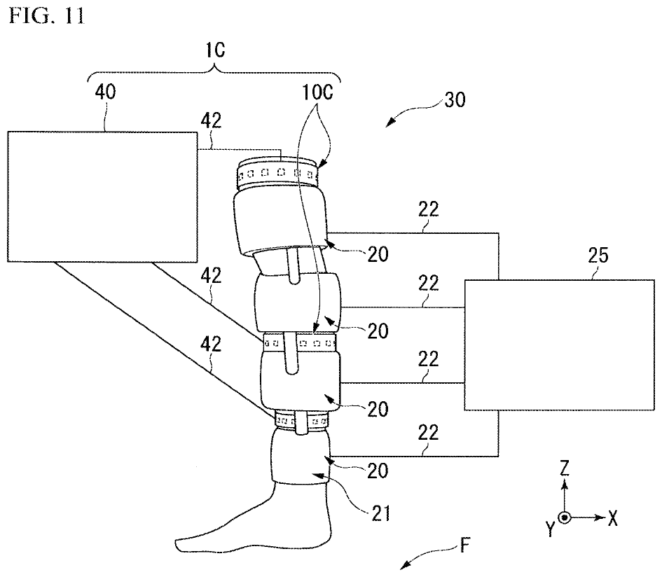

As illustrated in FIG. 11, the in-vivo measurer 1C includes sensors 10C for electrical impedance tomography and an electric controller 40. A current or a potential difference is applied to the subject by using the sensor 10C for electrical impedance tomography, and thereby an internal structure of the subject can be visualized. In addition, a temporal change of visualized internal biological information (for example, a conductivity distribution) is observed, and thereby it is possible to ascertain a change in distribution of interstitial fluid, lymphatic fluid, and venous blood.

The in-vivo measurer 1C has preferably two or more sensors 10C for electrical impedance tomography. The in-vivo measurer 1C includes two or more sensors 10C for electrical impedance tomography, and thus the flow of interstitial fluid, lymphatic fluid, and venous blood can be ascertained. As the number of the sensors 10C for electrical impedance tomography increases, the flow of interstitial fluid, lymphatic fluid, and venous blood can be ascertained more accurately, and thus the upper limit of the number of the sensors 10C for electrical impedance tomography is not particularly limited.

When the subject is wearing the sensors 10C for electrical impedance tomography, the in-vivo measurer 1C applies a predetermined current or potential difference between the electrodes 15 and measures a potential difference or current. In the case where the current is applied, a potential difference is measured based on a predetermined current application/voltage measurement pattern (pattern in which two electrodes are sequentially selected from a large number of electrodes, the current is applied, and the potential difference is sequentially measured). At this time, it is desirable to also measure a phase (a temporal difference between the applied current and the measured potential difference). In the case where the potential difference is applied, a current is measured based on a predetermined voltage application/current measurement pattern (pattern in which two electrodes are sequentially selected from a large number of electrodes, the potential difference is applied, and the current is sequentially measured). At this time, it is preferable to also measure a phase (a temporal difference between the applied potential difference and the measured current).

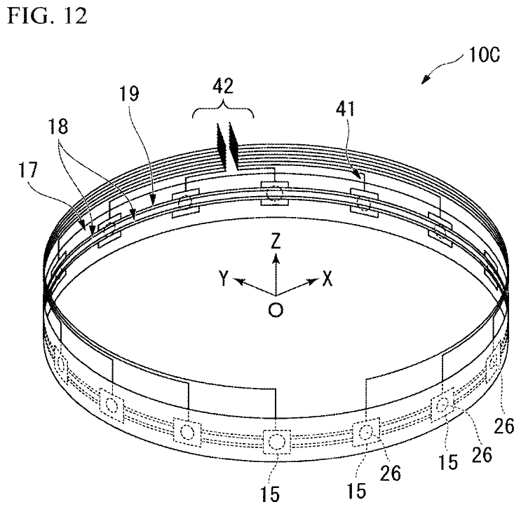

The in-vivo measurer 1C measures coordinates of contour measurement points 26 illustrated in FIG. 12 using the sensors 10C for electrical impedance tomography. The obtained information such as the coordinates of the contour measurement points 26 is sent to the contour estimator 2 of the measurement calculator SOC.

### Sensor for Electrical Impedance Tomography

As illustrated in FIG. 12, the sensor 10C for electrical impedance tomography includes four or more electrodes 15 (the number Q of electrodes), a stretch sensor 18 that measures displacement in an extending direction, a bend sensor 19 that measures displacement in a bending direction, and a support 17 that holds the electrodes 15, the stretch sensor 18, and the bend sensor 19. As illustrated in FIG. 12, for example, the stretch sensor 18 and the bend sensor (angle detection sensor) 19 are arranged at four or more contour measurement points 26 on the support 17. Since the sensors 10 for electrical impedance tomography includes a coordinate measurement means capable of measuring coordinate positions of the electrodes 15, such as the stretch sensor 18 and the bend sensor 19, the Jacobian matrix calculator 3 can calculate the Jacobian matrix even without measuring the position of each electrode 15 in advance.

The stretch sensor 18 is a stretchable strain sensor. The stretch sensor 18 measures displacement in a stretching/contracting direction near the contour measurement points 26 when the subject is wearing the support 17 having the sensors 10C for electrical impedance tomography. In the fourth embodiment, the contour measurement points 26 are set as arrangement positions of the electrodes 15.

The bend sensor 19 is a sensor capable of measuring angular displacement. The bend sensor 19 measures displacement in the bending direction near the contour measurement points 26 when the subject is wearing the sensors 10C for electrical impedance tomography.

### Contour Estimator

The contour estimator 2 obtains, as reference points, coordinates (x, y) of the contour measurement points 26 before the subject wears the sensors 10C for electrical impedance tomography, and estimates a leg contour 852 of the subject from a change in the coordinates (x, y) of the contour measurement points 26 when the subject is wearing the sensors 10C for electrical impedance tomography. The stretch sensor 18 and the bend sensor 19 are preferably disposed at the same positions as the respective electrodes 15, but may be disposed at respective independent positions regarding an x position and a y position, or may be disposed near the coordinates (x, y) of the electrodes 15. By using data from the stretch sensor 18 and the bend sensor 19, the contour estimator 2 estimates a two-dimensional leg contour ∂Ω of the subject for each sensor 10C for electrical impedance tomography from the x coordinate and the y coordinate of each contour measurement point 26 in the sensor 10C for electrical impedance tomography. In order to obtain the leg contour ∂Ω of the subject from the coordinate data of the contour measurement points 26, the contour estimator 2 performs interpolation between the contour measurement points 26 (between individual coordinate points). The contour estimator 2 estimates the contour ∂Ω of the subject more accurately using the position coordinates of the contour measurement points 26 and an interpolation curve such as a B-spline curve. As the interpolation curve, a Bézier curve, a Lamé curve, or the like may be used in addition to the B-spline curve. The position of the q-th electrode 15 is represented by a length r_{q} from a n electrode position to the center (origin) O of the sensor 10C for electrical impedance tomography and an angle θ formed by a line connecting the position of the electrode 15 and the origin O) and the x axis. The obtained information of the contour ∂Ω of the subject is sent to the Jacobian matrix calculator 3C.

### Jacobian Matrix Calculator

The Jacobian matrix calculator 3C uses predetermined current application/voltage measurement patterns (or voltage application/current measurement patterns) of the electrodes 15 and coordinates of the electrodes 15 and the leg contour ∂Ω of the subject (mesh coordinates obtained by dividing the leg contour ∂Ω) estimated by the contour estimator 2 to calculate the Jacobian matrix J* of the internal structure Ω of the subject (* is a symbol meaning that estimation has been performed for the subject). The Jacobian matrix calculator 3C sends the Jacobian matrix J of the subject to the electrical property distribution calculator 4. In a method of calculating the Jacobian matrix, calculation can be performed by a method similar to that of the Jacobian matrix calculator 3.

The fourth embodiment has been described above. In the fourth embodiment, the sensors 10C for electrical impedance tomography can measure the coordinates of the electrodes 15, and the contour estimator 2 can estimate the contour of the subject. Therefore, it is not necessary to measure the contour of the subject, and it is possible to cope with daily variations in the contour of the subject.

In the fourth embodiment, the contour ∂Ω of the subject is estimated by using the stretch sensor 18 and the bend sensor 19, but only one of the stretch sensor 18 and the bend sensor 19 may be used. In addition, for example, the leg contour ∂Ω of the subject may be simply estimated by using the support 17 having a standard size such as S, M, or L.

### Fifth Embodiment

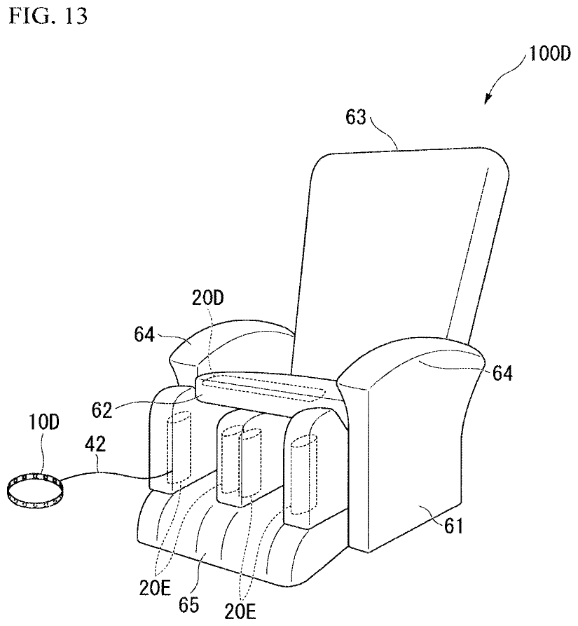

Next, a massage apparatus 100D according to a fifth embodiment will be described. As illustrated in FIG. 13, the massage apparatus 100D is a chair type massage apparatus.

The massage apparatus 100D includes a base 61 that stands on a floor surface and supports an entire chair, a seat 62 that supports the buttocks of the subject on an upper side of the base 61, a backrest 63 that is disposed on a rear side of the seat 62 and supports the back of the subject, armrests 64 that support the elbows of the subject on both sides of the seat 62, and a leg support 65 that is disposed on a front side of the seat 62 and supports the legs of the subject. The electric controller 40, the pressing controller 25, the measurement calculator 50, and the like of the in-vivo measurer 1 are disposed inside the massage apparatus 100D (for example, under the seat 62).

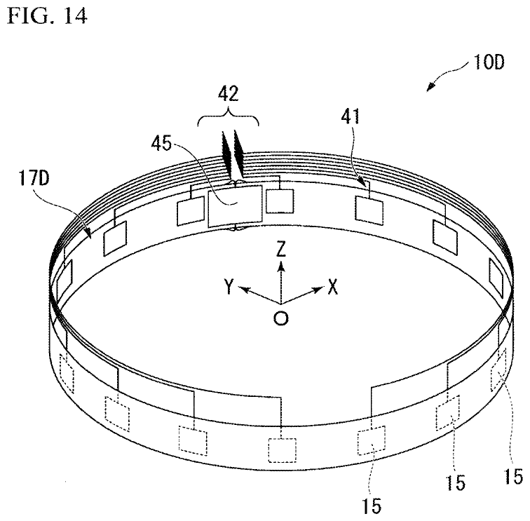

The massage apparatus 100D includes sensors 10D for electrical impedance tomography connected to the electric controller 40 inside the massage apparatus 100D via an electric wire bundle 42. As illustrated in FIG. 14, a support 17D of the sensors 10D for electrical impedance tomography may have a band shape, or in a case where it is difficult to cover the entire perimeter of a leg, the support may have only a partial band shape in a circumferential direction, instead of the band shape. In addition, the support 17D of the sensors 10D for electrical impedance tomography has a detachable portion 45 to be easily attached and detached. The detachable portion 45 is, for example, a hook-and-loop fastener. Since the detachable portion 45 is provided, the subject can easily wear the sensors 10 for electrical impedance tomography on a region where the subject wants to have the effect of the massage.

The massage apparatus 100D includes a presser 20D for a thigh and a presser 20E for a calf. By arranging the pressers 20D and 20E in this manner, the interstitial fluid, the lymphatic fluid, and the venous blood can recirculate while the subject sits in the chair.

The fifth embodiment has been described above. The massage apparatus 100D of the fifth embodiment enables the interstitial fluid, the lymphatic fluid, and the venous blood to recirculate while the subject sits in the chair. In addition, in the massage apparatus 100D, a region where the subject can freely check the effect of the massage can be set.

The massage apparatus according to the present embodiment has been described above. Note that the technical scope of the present invention is not limited to the above embodiments, and various modifications can be made without departing from the gist of the present invention. Additionally, it is possible to appropriately replace the components in the above-described embodiments with well-known components without departing from the gist of the present invention, and the above-described embodiments may be appropriately combined.

### EXAMPLE

Next, an example of an experiment for verifying the effectiveness of the massage apparatus of the present disclosure will be described.

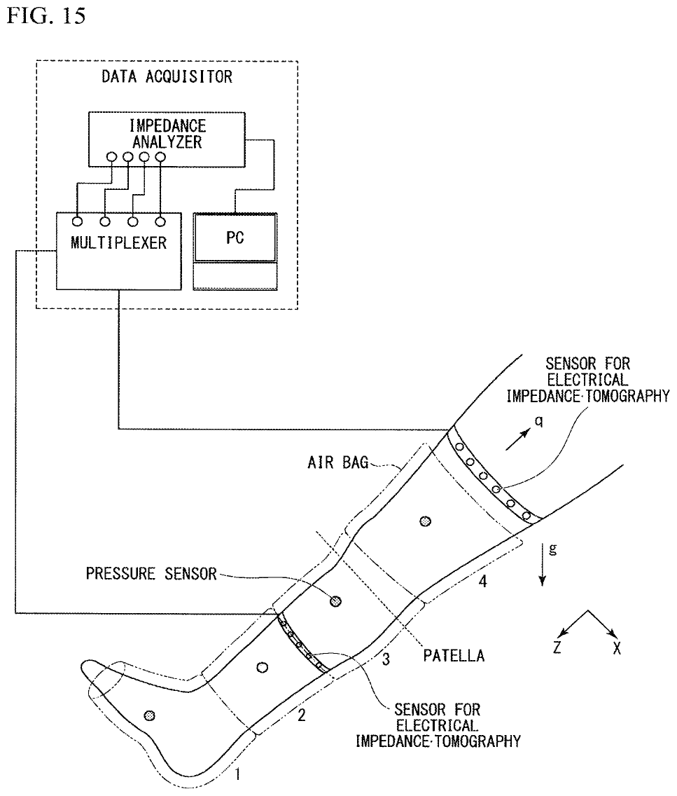

FIG. 15 illustrates an example of a massage apparatus used in the present example. The massage apparatus includes a sleeve having four air bags, pressure sensors that measure pressure in respective portions, and sensors for electrical impedance tomography provided on a thigh part and a calf part. Here, 16 electrodes were arranged in each sensor for electrical impedance tomography.

A healthy male (age: 30) was set as a subject, and recirculation of interstitial fluid, lymphatic fluid, and venous blood of muscles and fat was imaged during massage by an intermittent pneumatic compression (IPC) mechanism using the massage apparatus of the present invention. The adjacent method was used for the electrical impedance tomography measurement. In addition, a time change in pressure in each of the air bags was recorded. A current was applied to a calf and a thigh of the subject with an applied current of 1 mA, a voltage was measured, and image reconstruction was performed by the above method. Specifically, conductivity distribution images of the calf and the thigh were reconstructed.

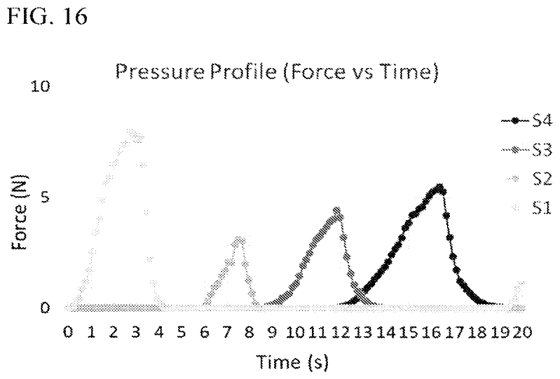

FIG. 16 illustrates a change in pressure in each air bag during massage. In FIG. 16, the vertical axis represents force (N), and the horizontal axis represents time(s). S1 denotes the pressure of an air bag (first air bag) in 1 of FIG. 15. S2 denotes the pressure of an air bag (second air bag) in 2 of FIG. 15. S3 denotes the pressure of an air bag (third air bag) in 3 of FIG. 15. S4 denotes the pressure of an air bag (fourth air bag) in 4 of FIG. 15. As illustrated in FIG. 16, pneumatic compression was intermittently performed for the massage by temporally controlling one cycle of pneumatic pressures of the four air bags and also spatially controlling the pneumatic pressures of the four air bags from an extremity side (toe side) toward the thigh.

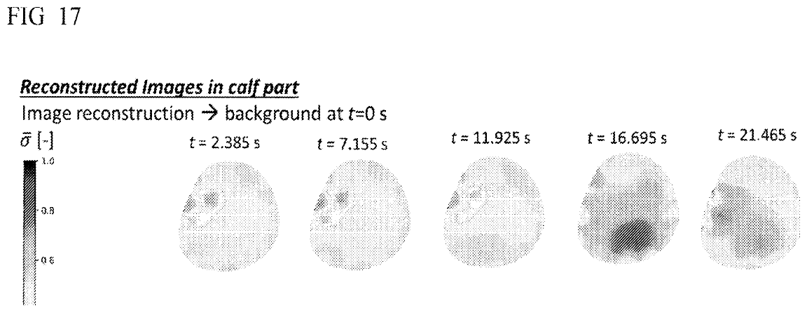

FIG. 17 illustrates a temporal change of a conductivity distribution obtained by the electrical impedance tomography measurement during the massage when a calf cross section of the subject's left foot is viewed from the head side. In the massage by the intermittent pneumatic compression (IPC) mechanism, since the change in pressure of each air bag in FIG. 16 has a time delay from the extremity side toward the thigh, the interstitial fluid, the lymphatic fluid, and the venous blood gradually recirculate from the extremity side toward the thigh, and spatial and temporal variations in recirculation can be observed as spatial and temporal variations in conductivity region size and color depth by the sensors for electrical impedance tomography. Incidentally, the interstitial fluid, the lymphatic fluid, and the venous blood are ionic liquids and have been found to have higher conductivity than fat, muscles, and bones. Specifically, in the case of this subject (the blue position in the upper left portion is the tibia and thus no special attention is attracted), conductivity around the gastrocnemius muscle (posterior muscle) gradually increased until around 11 seconds, conductivity around not only the gastrocnemius muscle but also the soleus muscle (muscle in the vicinity of the lower center portion) increased until around 16 seconds, and thereafter, the conductivity around the gastrocnemius muscle and the soleus muscle slightly decreased and the conductivity around the posterior tibial muscle (blue muscle in the upper left portion in the vicinity of the tibia) slightly increased until around 21 seconds. That is, the time and spatial position of the interstitial fluid, the lymphatic fluid, and the venous blood which recirculate can be ascertained.

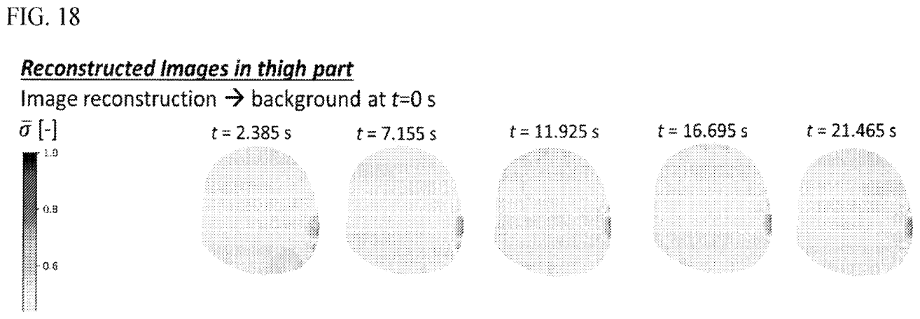

FIG. 18 illustrates a temporal change of a conductivity distribution obtained by the electrical impedance tomography measurement during the massage when a thigh of a subject's left foot is viewed from the head side. In particular, in this thigh, the image is shown for a time of up to 21 seconds, but the interstitial fluid, the lymphatic fluid, and the venous blood do not yet recirculate from the calf on which the massage is performed to the thigh, and there is no significant change in the image particularly at this time. However, as will be described below, after about 170 seconds, it can be seen that the interstitial fluid, the lymphatic fluid, and the venous blood recirculated and reached the thigh from the calf on which the massage is performed. As illustrated in FIGS. 17 and 18, it has been described that the continued massage caused a spatial and temporal variations in conductivity distribution of the calf part and a thigh part. In addition, a difference in spatial and temporal variations in conductivity distribution between the calf part and the thigh part is observed.

As described above, it has been described that the massage apparatus of the present disclosure has sufficient performance for detecting a physiological response of the recirculation of the interstitial fluid, the lymphatic fluid, and the venous blood of muscles and fat during massage. It was verified through experiments in subjects that the recirculation of the interstitial fluid, the lymphatic fluid, and the venous blood of muscles and fat in a reconstructed image can be sufficiently distinguished.

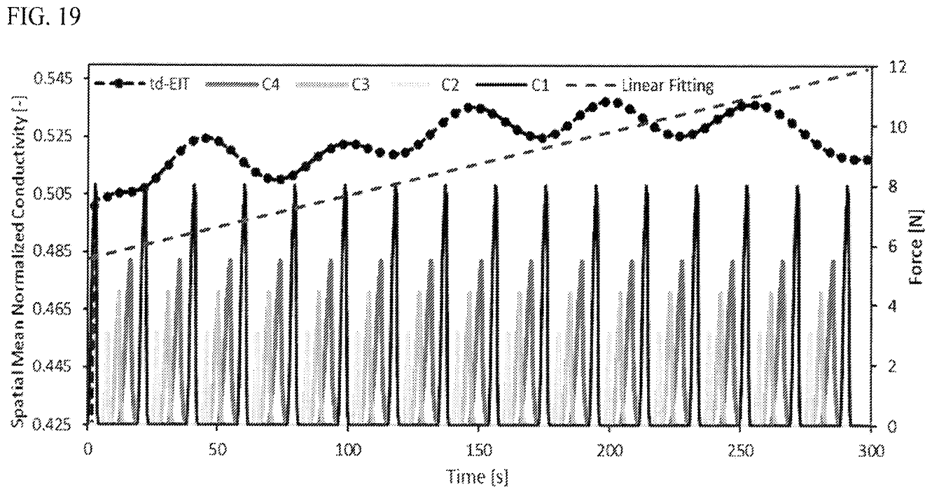

Further, the spatial mean normalized conductivity <σ> was defined, the magnitude of the conductivity change during massage was quantified, and the recirculation of the interstitial fluid, the lymphatic fluid, and the venous blood was evaluated in detail. FIG. 19 illustrates the spatial mean normalized conductivity <σ> in the calf part and a relationship between the pressure and the time of each air bag. The horizontal axis in FIG. 19 represents a time(s), the vertical axis on the left side in FIG. 19 represents spatial mean normalized conductivity, and the vertical axis on the right side thereof represents force (N). Here, td-EIT denotes the spatial mean normalized conductivity <σ> in the calf part. C1 in FIG. 19 denotes the pressure of the first air bag. C2 in FIG. 19 denotes the pressure of the second air bag. C3 in FIG. 19 denotes the pressure of the third air bag. C4 in FIG. 19 denotes the pressure of the fourth air bag. Linear fitting represents a result of linear approximation of the spatial mean normalized conductivity <σ> and (td-EIT).

In the calf part, as compared with a periodic waveform of the pneumatic pressure, the spatial mean normalized conductivity <σ> increased with a long period and then tended to be constant, and the recirculation of the interstitial fluid, the lymphatic fluid, and the venous blood was observed every time one cycle of the pneumatic pressure ended. In particular, in the case of the subject. there is a tendency that the spatial mean normalized conductivity <σ> gradually increases while a gentle cycle is repeated before 260 seconds, and the spatial mean normalized conductivity <σ> gradually becomes constant while a gentle cycle is repeated after 260 seconds, and it can be seen that the recirculation is sufficiently performed in about 260 seconds.

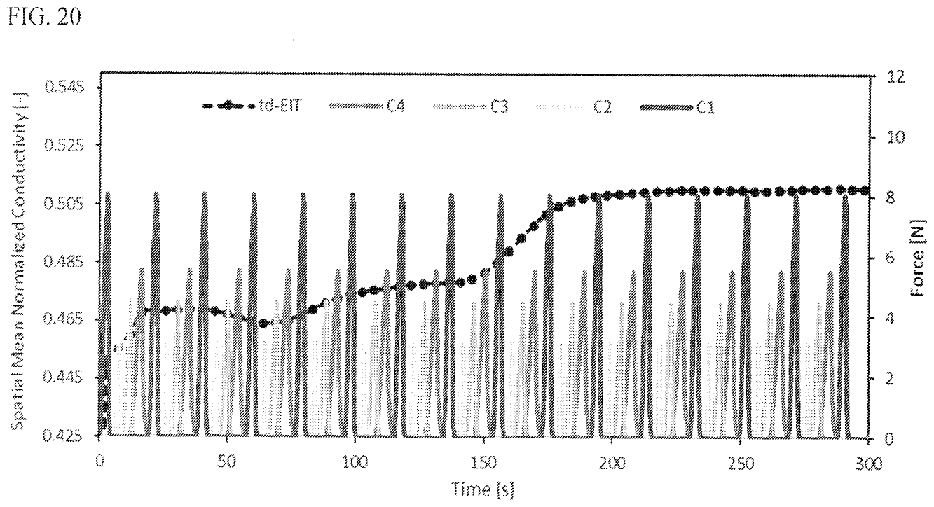

FIG. 20 is a graph illustrating spatial mean normalized conductivity <σ> in the thigh and a relationship between pressure in each chamber and a time. The horizontal axis in FIG. 20 represents a time(s), the vertical axis on the left side in FIG. 20 represents spatial mean normalized conductivity, and the vertical axis on the right side thereof represents force (N). The spatial mean normalized conductivity <σ> denoted by td-EIT indicates the spatial mean normalized conductivity in the calf part. C1 in FIG. 20 denotes the pressure of the first air bag. C2 in FIG. 20 denotes the pressure of the second air bag. C3 in FIG. 20 denotes the pressure of the third air bag. C4 in FIG. 20 denotes the pressure of the fourth air bag. In the thigh, unlike the calf on which the massage is performed, a tendency is observed in which the spatial mean normalized conductivity <σ> increases and becomes constant without a cycle with the lapse of time. In the case of the subject, until about 60 seconds, the interstitial fluid, the lymphatic fluid, and the venous blood do not yet recirculate from the calf on which the massage is performed to the thigh, and there is no significant change particularly at this time. It can be seen that the spatial mean normalized conductivity <σ> gradually increases after 60 seconds, rapidly increases after about 150 seconds, and then becomes constant after 170 seconds, and the interstitial fluid, the lymphatic fluid, and the venous blood sufficiently recirculate due to the massage on the calf in about 170 seconds. Note that the spatial mean normalized conductivity is conductivity obtained by spatial mean normalization of a conductivity distribution obtained by electrical impedance tomography.

As described above, according to the massage apparatus of the present disclosure, it has been confirmed that temporal and spatial variations in distribution of the interstitial fluid, the lymphatic fluid, and the venous blood in a living body due to the massage can be measured.

### REFERENCE SIGNS LIST

1 In-vivo measurer
2 Contour estimator
3 Jacobian matrix calculator
4 Electrical property distribution calculator
10 Sensor for electrical impedance tomography
20 Presser
21 Air bag
22 Flow channel
25 Pressing controller
40 Electric controller
42 Electric wire bundle
50 Measurement calculator 50
100 Massage apparatus

1. A massage apparatus comprising:
a plurality of pressers that press a subject; and
an in-vivo measurer that measures a change in biological information of the subject due to pressing, wherein
the in-vivo measurer has one or more sensors for electrical impedance tomography, and each of the sensors for electrical impedance tomography has four or more electrodes.

2. The massage apparatus according to claim 1, comprising two or more of the sensors for electrical impedance tomography.

3. The massage apparatus according to claim 1, comprising the pressers between the sensors for electrical impedance tomography.

4. The massage apparatus according to claim 1, wherein each of the pressers has one air bag.

5. The massage apparatus according to claim 1, wherein each of the pressers includes two or more air bags, and the air bags are capable of applying respective different magnitudes of pressure.

6. The massage apparatus according to claim 1, wherein the electrodes are arranged at uniform intervals.

7. The massage apparatus according to claim 1, wherein the in-vivo measurer applies a current or a potential difference between the electrodes, measures a potential difference and a phase based on a current application/voltage measurement pattern in a case where the current is applied, and measures a current and a phase based on a voltage application/current measurement pattern in a case where the potential difference is applied between the electrodes.

8. The massage apparatus according to claim 7, further comprising:
a Jacobian matrix calculator that calculates a Jacobian matrix of the subject based on the predetermined current application/voltage measurement pattern or voltage application/current measurement pattern, mesh coordinates obtained by dividing a contour of the subject, and coordinates of the respective electrodes; and
an electrical property distribution calculator that calculates an electrical property distribution that is the biological information from the Jacobian matrix of the subject calculated by the Jacobian matrix calculator and the potential difference and the phase or the current and the phase measured by the in-vivo measurer.

9. The massage apparatus according to claim 8, wherein the Jacobian matrix calculator calculates the Jacobian matrix by using machine learning.

10. The massage apparatus according to claim 8, further comprising a pressing controller that controls a pressure of each of the pressers based on the electrical property distribution.

11. The massage apparatus according to claim 10, wherein
the plurality of pressers are arranged at intervals in a longitudinal direction of a certain part of the subject, between the sensors for electrical impedance tomography, and
the pressing controller controls, in a case where a temporal change of the electrical property distribution is smaller than a predetermined value, a pressure of the presser adjacent to the sensor for electrical impedance tomography which obtains the electrical property distribution with a small temporal change.

12. The massage apparatus according to claim 11, wherein
the presser includes two or more of the air bags arranged in a circumferential direction of the certain part of the subject, the presser being configured to apply respective different magnitudes of pressure to each of the air bags, and
the pressing controller controls, in a case where the temporal change of the electrical property distribution is smaller than a predetermined value, a pressure of each of the air bags in the presser adjacent to the sensor for electrical impedance tomography which obtains the electrical property distribution with a small temporal change.
